(19) United States
(12) Patent Application Publication
(10) Pub. No.: US 2002/0193726 A1
(43) Pub. Date: Dec. 19, 2002

(54) Tampon applicator

(76) Inventor: Hugo Cimber, Bern (CH)

Correspondence Address: J George Seka, Townsend & Townsend & Crew, 8th Floor, Two Embarcadero Center, San Francisco, CA, 94111-3834, US

(21) Appl. No.: 10/181,297

(22) PCT Filed: Jan. 18, 2001
(86) PCT No.: PCT/CH01/00040

(30) Foreign Application Priority Data
Jan. 18, 2000 (CH) 89/00

(51) Int. Cl. A61F 13/20

(52) U.S. Cl. 604/11; 604/12; 604/14; 604/15

(57) ABSTRACT

A tampon applicator (1) consists of an insertion sleeve (2) in which a tampon (10) is introduced. Slidingly affixed within the insertion sleeve (2) is an ejection sleeve (4) for ejecting the tampon (10). In the insertion sleeve (2), a medicinal agent, which is present in the form of a pill (9), is disposed in such a way that this pill (9) is ejectable together with the tampon (10) by means of the ejection sleeve (4) through the leading end of the insertion sleeve (2).

The present invention relates to a tampon applicator according to the generic clause of patent claim 1.

The use of tampons having a medicinal agent are applied in particular in order to avoid a pH shift into the alkaline range in the vaginal environment and thus lessened resistance to infection. For this purpose, preparations containing particularly lactic acid and/or lactobacilli are used as medicinal agents.

Such tampon applicators are known. Thus, for example, EP-A-0 749 741 shows a respective device in which a closed chamber can be disposed in the insertion sleeve in front of the tampon head, which takes place for example by putting on or screwing on an additional container filled with the respective medicinal agent which is present in liquid form. The chamber formed by the container is closed toward the insertion sleeve by a film, whereas the other side is provided with a removable cover.

Prior to insertion or during insertion of the tampon into the vagina, the medicinal agent contained in the chamber is supposed to come in contact with the tampon situated in the insertion sleeve. The film of the container facing the tampon head must be broken open, which is supposed to take place by pushing the tampon forward by means of the ejection sleeve, for instance during the insertion of the tampon. For facilitating the breaking open of the film, mechanical means are provided.

Disadvantageous in this device is that the medicinal agent is accommodated in an additional closed container which must be put on the insertion sleeve prior to use. For this purpose, the respective connection means must be provided both on the container and on the insertion sleeve. The manufacture of the container and the filling of the medicinal agent with the respective closures thereby becomes relatively extravagant and correspondingly expensive. The same also applies to the insertion sleeve, which must be adapted for receiving the container.

Further, for example, U.S. Pat. No. 4,318,405 shows a tampon applicator in which, in the tip of the tampon, a cylindrical depression is made in which a capsule is inserted containing the medicinal agent. So that this capsule cannot fall out, it is provided that it be fastened in the tampon with an adhesive substance.

This tampon applicator has the disadvantage that through making the depression in the tampon and inserting the capsule in this depression, particularly if an adhesive substance is used, the manufacture becomes expensive. In the case of fabrication by machine, complicated units are necessary for this purpose.

Now the task of the above invention consists in creating a tampon applicator in which the aforesaid disadvantages are eliminated, which makes possible simple and favorable producing of the individual components and simple assembly, and which is applicable in simple form.

According to the invention, the solution of this task takes place by means of the features recited in claim 1.

Through the possibility that the medicinal agent can be made in pill form, which pill can be inserted between insertion sleeve and tampon, the manufacture and the application of the tampon applicator is substantially facilitated. The pill, which may be obtained by pressing of a powdery preparation, contains the lactic acid and/or the lactobacilli, for instance. The pill thus formed then dissolves in the vagina, and the medicinal agents can fully develop their effect.

One advantage of the invention consists in that the pill is accommodated in the insertion sleeve in such a way that it comes to lie between the tip of the tampon and the leading end of the insertion sleeve. A simple assembly of the tampon applicator can be achieved thereby, it being

. . . simple assembly of the tampon applicator [can be] achieved thereby, it being possible in particular to use an ordinarily usable tampon unchanged. For this purpose, the insertion sleeve may be held substantially vertically with the opening upward and the leading end downward. The pill can be introduced into the insertion sleeve by simple dropping, it independently aligns itself in the tip of the insertion sleeve. Thereafter, the tampon may be put in and the ejection sleeve introduced. This operation can be quickly carried out by simply constructed machines.

By affixing holding means to the insertion sleeve, the pill can be held in the condition introduced into the insertion sleeve, whereby it is achieved that upon introduction of the tampon into the insertion sleeve, the pill remains at the predetermined location even if the insertion sleeve must be moved for that purpose.

An advantageous design of the invention consists in that the insertion sleeve and the ejection sleeve are each formed of a tubular body made of cardboard or a cardboard-like material which is coated on at least one side, whereby simple and economical manufacture can be achieved. Advantageously, the leading end of the insertion sleeve is provided with a cap-like closure part which is formed by tongue-shaped sections, each having substantially the shape of a spherical wedge, and which are expandable upon ejection of the tampon together with the pill. This tampon applicator can thereby be inserted in the vagina without difficulty, the ejection of the tampon together with the pill is achieved without difficulty.

When a pill is used whose two surfaces exhibit the shape of a cap and which is consequently symmetrical, the introduction of this pill into the insertion sleeve is particularly simple since the pill can be put into the insertion sleeve in any desired position and is aligned therein. The introduction of the pill into the insertion sleeve is particularly simple when the pill has the shape of a sphere.

Advantageously, the surface of the pill directed toward the tip of the tampon is formed as a concave surface which substantially coincides with the shape of the tip of the tampon. An ordinarily usable tampon need not be changed. An optimum seating of the pill on the tampon is thereby achieved, insertion of the tampon together with the pill is possible without difficulty.

A further embodiment may have a tampon with a recess made in the area of the tip of the tampon. Through the use of a pill whose two surfaces have the shape of a cap, the pill is accommodated optimally in the tampon applicator. Insertion of the tampon together with the pill into the vagina is optimally possible.

Advantageously, the tampon applicator provided with a tampon and the pill is always arranged with a hermetic cover which can be torn open and removed prior to use of the tampon applicator. The requirements of hygiene are thereby fulfilled, the pill remains in the desired condition for a considerable time.

Exemplary embodiments of the present invention are explained in detail below, by way of example, with the aid of the enclosed drawing.

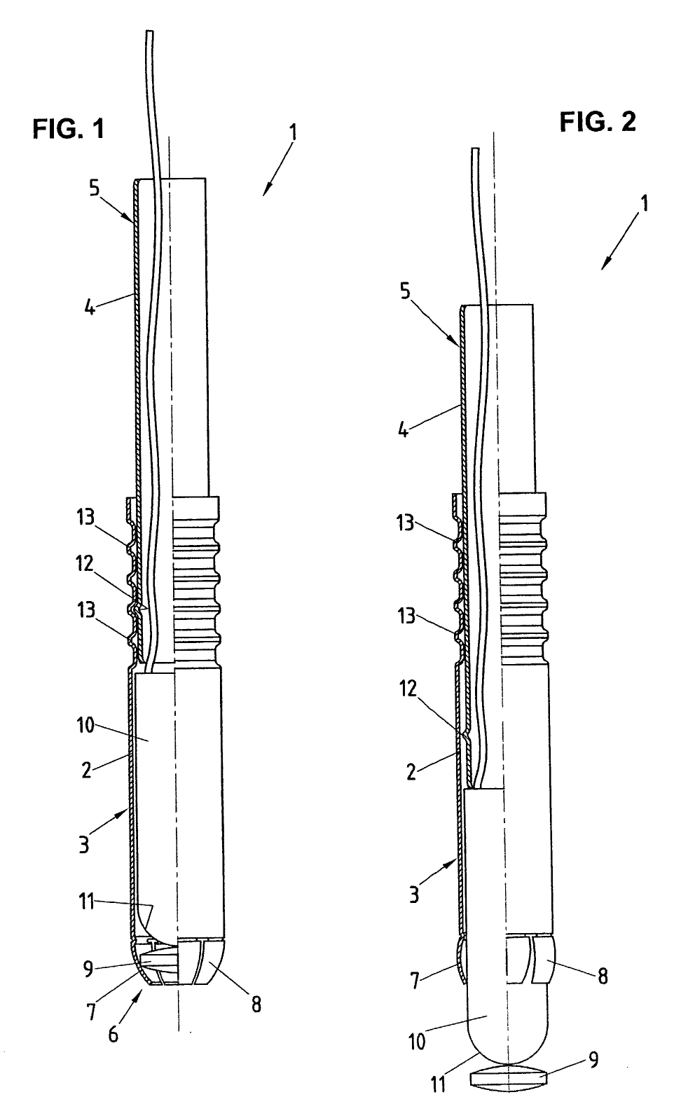

FIG. 1 shows a view of a first embodiment of a tampon applicator according to the invention in its original condition, partially in section;

FIG. 2 shows a view of the tampon applicator according to FIG. 1 during the ejection of the tampon together with the introduced pill, partially in section; and

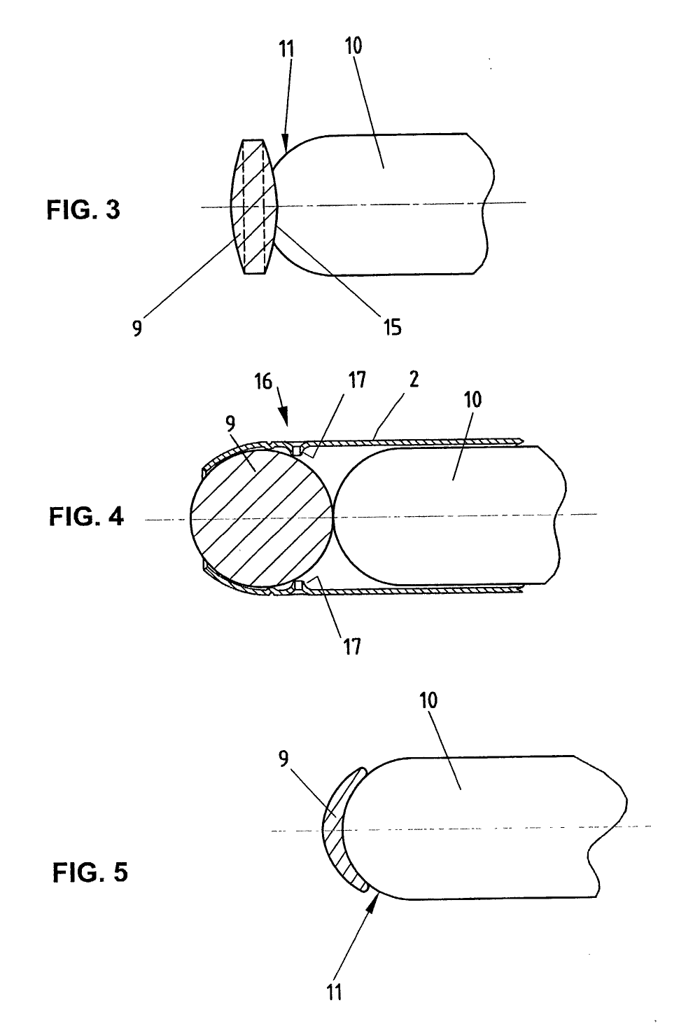

FIGS. 3 to 5 show further shapes of the pill and its arrangement relative to the tampon in the insertion sleeve. shaped sections 8 each have substantially the shape of a spherical wedge and are expandable, as will be described later on.

Placed in the insertion sleeve 2 is a pill 9 which comes to lie in the inner region of the cap-shaped closure portion 7 of the insertion sleeve 2. Furthermore, a tampon 10 is introduced into the insertion sleeve 2 so that the pill 9 is held in the insertion sleeve 2 between the tip 11 of the tampon 10 and the inside of the cap-shaped closure portion 7. Advantageously, the pill 9 has a convexly curved surface which is substantially adapted to the cap-like closure portion 7 and rests against the latter. The surface directed toward the tip 11 of the tampon 10 is likewise formed convexly corresponding to the opposite surface in this exemplary embodiment.

The ejection sleeve 4 is pushed into the insertion sleeve 2 until its inner end region butts against the tampon 10, the ejection sleeve 4 being provided, for holding this position fast, with a nub 12 which can snap into one of the grooves 13 correspondingly made on the insertion sleeve 2.

The insertion sleeve 2 and the ejection sleeve 4 are made of a flexible material, for example cardboard. This cardboard is provided with a coating having at least a certain resistance to water. This coating may be omitted on the inside of the ejection sleeve 4. Of course, other suitable materials are also conceivable, for instance on a plastics basis.

In this state, the tampon applicator 1 is packed in a manner known per se in a non-depicted, hermetic cover.

Prior to use of this tampon applicator 1, the cover is removed, the tampon applicator 1 may be inserted into the vagina. The ejection sleeve 4 is then pushed into the insertion sleeve 2. The tampon 10 pressed with the pill 9 against the tongue-shaped sections 8, which are expanded, which is made optimally possible by the cap-like shape of the corresponding surface of the pill. The tampon 10 is ejected with the pill 9 from the insertion sleeve, as shown in FIG. 2.

As shown in FIG. 3, a recess 15 may be made in the tip 11 of the tampon 10. Into this recess 15, the convex surface of the pill 9 directed toward the tip 11 of the tampon 10 comes to lie, this pill 9 being designed the same as the pill 9 shown in FIGS. 1 and 2.

From FIG. 4 it is apparent that the pills 9 shown in FIGS. 1 and 2 may also have the shape of a sphere. In the front region of the insertion sleeve 2, shown here in section, holding means 16 are visible which hold the pill 9 introduced into the insertion sleeve 2 in the predetermined position. In the example depicted, the holding means 16 consist of two impressions 17 which project inwardly. Thereby, the insertion sleeve 2 may be moved before or during the introduction of the pill without the pill 9 changing its position. Of course, such holding means 16 may also be affixed when pills 9 having other shapes are used, for instance with those shown in the other figures.

Also conceivable here is that the tampon 10 could be provided with a recess which corresponds, say, to the one shown in FIG. 3.

FIG. 5 shows the tip 11 of the tampon 10 on which the pill 9 is placed. The surface facing the tip 11 of the tampon is correspondingly formed concavely, whereas the opposite surface, which is directed toward the leading end of the tampon applicator, has the shape of a cap. This pill 9 thus rests cap-like on the tip 11 of the tampon 10 upon ejection of the tampon.

By means of these embodiments, it is ensured that the respective pill 9 can be optimally positioned in the vagina together with the tampon 10 and that the medicinal effect can develop.

1. Tampon applicator (1) having an insertion sleeve (2) in which a tampon (10) is introduced, having an ejection sleeve (3) [sic] slidable within the insertion sleeve (2) for ejecting the tampon (10), and having a medicinal agent which takes the form of a pill and is ejectable together with the tampon (10) through the leading end (6) of the insertion sleeve (2), characterized in that the pill (9) is placed in the insertion sleeve (2) in such a way that it comes to lie between the tip (11) of the tampon (10) and the leading end (6) of the insertion sleeve (2) and that affixed to the insertion sleeve (2) are holding means (16) for the pill (6) [sic], which consist of at least one inwardly projecting impression (17) made in the insertion sleeve (2), by means of which the pill (6) is held in the condition placed in the insertion sleeve (2).

2. Tampon applicator (1) according to claim 1, characterized in that the insertion sleeve (2) and the ejection sleeve (3) are each formed of a tubular body (3, 5) of cardboard or a cardboard-like material which is coated on at least one side, and that the leading end (6) of the insertion sleeve (2) is provided with a cap-like closure portion (7) formed by tongue-shaped sections (8) which each have substantially the shape of a spherical wedge and which are expandable upon ejection of the tampon (10) together with the pill (9).

3. Tampon applicator (1) according to claim 1 or 2, characterized in that at least the surface of the pill (9) directed toward the leading end (6) of the tampon (10) has the shape of a cap.

4. Tampon applicator (1) according to claim 3, characterized in that the surface of the pill (9) directed toward the leading end (6) of the insertion sleeve (2) and the surface directed toward the tip (11) of the tampon (10) each have the shape of a cap.

5. Tampon applicator (1) according to claim 3 or 4, characterized in that the pill (9) has the shape of a sphere.

6. Tampon applicator (1) according to claim 3, characterized in that the surface of the pill (9) directed toward the tip (11) of the tampon (10) has a concave shape and substantially coincides with the shape of the tip (11) of the tampon (10).

7. Tampon applicator (1) according to claim 4 or 5, characterized in that made in the area of the tip (11) of the tampon (10) is a recess (15) which corresponds to the shape of the cap of the pill (9).

8. Tampon applicator (1) according to one of the claims 1 to 7, characterized in that the pill (6) is formed by pressing of a powdery preparation.

9. Tampon applicator (1) according to one of the claims 1 to 8, characterized in that the tampon applicator (1) is provided with a hermetic cover which can be torn open and removed prior to use of the tampon applicator (1).
